(19) United States
(12) Patent Application Publication
(10) Pub. No.: US 2025/0279113 A1
(43) Pub. Date: Sep. 4, 2025

(54) AI-BASED DISEASE DIAGNOSIS METHOD AND AN APPARATUS USING VOICE DATA

(71) Applicant: Grace Jung Eun SHIN, Seoul (KR)

(72) Inventor: Grace Jung Eun SHIN, Seoul (KR)

(21) Appl. No.: 18/859,067

(22) PCT Filed: Apr. 25, 2023
(86) PCT No.: PCT/KR2023/005634

§ 371 Date: Oct. 22, 2024

(30) Foreign Application Priority Data
Apr. 25, 2022 (KR) 10-2022-0050564

(51) Int. Cl. G10L 25/66; A61B 5/00; G10L 21/02; G10L 25/27; G10L 25/60

(52) U.S. Cl. CPC G10L 25/66; A61B 5/4803; A61B 5/7203; A61B 5/7221; G10L 21/02; G10L 25/27; G10L 25/60

(57) ABSTRACT

The present disclosure provides an AI-based disease diagnosis method using voice data, the method including collecting first voice data, generating second voice data by removing at least one of noise, unique characteristics of voice, or disease characteristics of voice from the first voice data, and diagnosing, based on AI, at least one disease of dementia, depression, or hearing loss, using the second voice data.

### TECHNICAL FIELD

The present disclosure relates to an AI-based disease diagnosis method and apparatus using voice data, and more particularly, to an AI-based method and apparatus for diagnosing dementia, depression, or hearing loss of a user using a voice data obtained by the user who performs a list of preset tasks.

### BACKGROUND ART

Dementia is an acquired disease that causes damage to cognitive function and changes in personality. As the aging population accelerates, the number of dementia patients is increasing, and the burden of dementia management and treatment is increasing day by day. Dementia is a disease that affects not only the patient but also the entire family's daily life, but early diagnosis is difficult and the pathogenesis is not clearly known, and thus there is no definitive treatment. Accordingly, a dementia diagnosis method is needed that can help diagnose dementia early using a simple method.

Meanwhile, depression refers to a state in which overall mental function is continuously deteriorated, which has an adverse effect on daily life. Depression is an unavoidable mental illness for modern people who are under a lot of stress, but there are psychological barriers to diagnosis and treatment, and thus many patients are delayed in diagnosing and treating the disease. Accordingly, a simple method to diagnose depression is needed.

A diagnostic method using voice data is proposed to easily diagnose these mental health-related diseases. The human voice has characteristics that are difficult to forge, and various pieces of information about human mental health and behavior through voice can be obtained, diseases may be diagnosed based on AI using this voice data.

### DESCRIPTION OF EMBODIMENTS

### Technical Problem

It is an objective of the present disclosure to provide an AI-based disease diagnosis method and apparatus using voice data, by which at least one disease of dementia, depression, or hearing loss is diagnosed based on AI using voice data.

It is an objective of the present disclosure to provide an AI-based disease diagnosis method and apparatus using voice data by which reliability of voice data is increased using not only voice data but also vision data including a face image.

### Solution to Problem

According to an embodiment of the present disclosure, an AI-based disease diagnosis method using voice data includes collecting first voice data, generating second voice data by removing at least one of noise, unique characteristics of voice, or disease characteristics of voice from the first voice data, and diagnosing, based on AI, at least one disease of dementia, depression, or hearing loss, using the second voice data.

In an embodiment, the disease diagnosis method further includes, collecting vision data including a face image of a user, determining reliability of the second voice data using the vision data, and removing a section in which the reliability of the second voice data is less than a preset value.

In an embodiment, the first voice data or the second voice data includes at least one piece of information of a type of voice, glottal attack, resonance, pitch, loudness, or voice quality or timbre.

In an embodiment, the first data is collected based on a list of set tasks to perform.

In an embodiment, the list of set tasks to perform includes making a long “ah” sound, and the unique characteristics of voice or disease characteristics of the voice is set based on the making of a long “ah” sound.

In an embodiment, the list of set tasks to perform includes making an “ipipi” sound, and the unique characteristics of voice or disease characteristics of the voice is set based on the making of an “ipipi” sound.

In an embodiment, the list of set tasks to perform includes at least one of counting backwards, describing pictures, reading scenarios, or reading newspaper editorials, and the at least one disease of dementia, depression, or hearing loss is diagnosed based on the list of set tasks to perform.

According to another embodiment of the present disclosure, an AI-based disease diagnosis apparatus using voice data includes a voice data collection unit that collects first voice data, and a control unit that generates second voice data by removing at least one of noise, unique characteristics of voice, or disease characteristics of voice from the first voice data, and diagnoses, based on AI, at least one disease of dementia, depression, or hearing loss, using the second voice data.

### Advantageous Effects of Disclosure

According to an embodiment of the present disclosure, diseases such as dementia, depression, or hearing loss may be easily diagnosed based on AI by using voice data including information related to individual's unique characteristics and mental health, thereby reducing the burden of treatment on patients and medical staff.

Furthermore, by removing unique characteristics and the like that are not related to the user's mental health from voice data, reliability of disease diagnosis may be improved, and by using vision data that includes face images as well as voice data, reliability of voice data may be further improved and accuracy in disease diagnosis may be improved.

### BRIEF DESCRIPTION OF DRAWINGS

FIG. 1 is a block diagram of an AI-based disease diagnosis apparatus using voice data according to an embodiment of the present disclosure.

FIG. 2 is a block diagram for describing a configuration of a control unit according to an embodiment of the present disclosure.

FIG. 3 is a diagram for describing a disease diagnosis method according to an embodiment of the present disclosure.

FIG. 4 shows an example of a list of preset tasks to perform according to an embodiment of the present disclosure.

FIG. 5 is a flowchart of an AI-based disease diagnosis method using voice data according to an embodiment of the present disclosure.

### MODE OF DISCLOSURE

Various embodiments of the disclosure will be described with reference to the accompanying drawings. However, it should be understood that the disclosure is not limited to these particular embodiments but also includes various modifications, equivalents, and/or alternatives thereof. Throughout the specification and drawings, like reference numerals may be used to denote like elements or components.

Terms such as “first,” “second,” “A,” and “B” are used herein merely to describe a variety of constituent elements, but the constituent elements are not limited by the terms. Such terms are used only for the purpose of distinguishing one constituent element from another constituent element. For example, without departing from the right scope of the disclosure, a first constituent element may be referred to as a second constituent element, and vice versa. The term “and/or” includes any and all combinations of one or more of the associated listed items.

Terms used in the specification are used for explaining a specific embodiment, not for limiting the disclosure. Thus, an expression used in a singular form in the specification also includes the expression in its plural form unless clearly specified otherwise in context. Also, terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

Unless defined otherwise, all terms used herein including technical or scientific terms have the same meanings as those generally understood by those of ordinary skill in the art to which the disclosure may pertain. The terms as those defined in generally used dictionaries are construed to have meanings matching that in the context of related technology and, unless clearly defined otherwise, are not construed to be ideally or excessively formal.

Hereinafter, embodiments of the present disclosure are described in detail with reference to the accompanying drawings.

In the following descriptions, “first voice data” refers to voice data that is collected from a voice data collection unit and is not processed specially, and “second voice data” refers to voice data that has undergone special processing for use in diagnosing diseases.

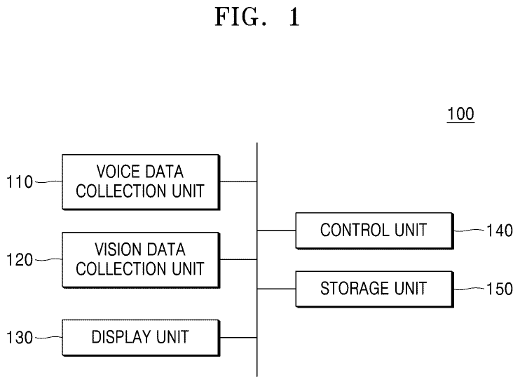

FIG. 1 is a block diagram of an AI-based disease diagnosis apparatus using voice data according to an embodiment of the present disclosure.

Referring to FIG. 1, an AI-based disease diagnosis apparatus 100 using voice data may include a voice data collection unit 110, a vision data collection unit 120, a display unit 130, a control unit 140, and a storage unit 150.

The voice data collection unit 110 may collect user's first voice data, convert the collected data into an input signal, and transmit the input signal to the control unit 140. The voice data collection unit 110 may be implemented by devices such as microphones, in detail, for soundproof facilities, by a nude microphone that is not processed with any voice processing, and for a noisy environment, by a directional microphone.

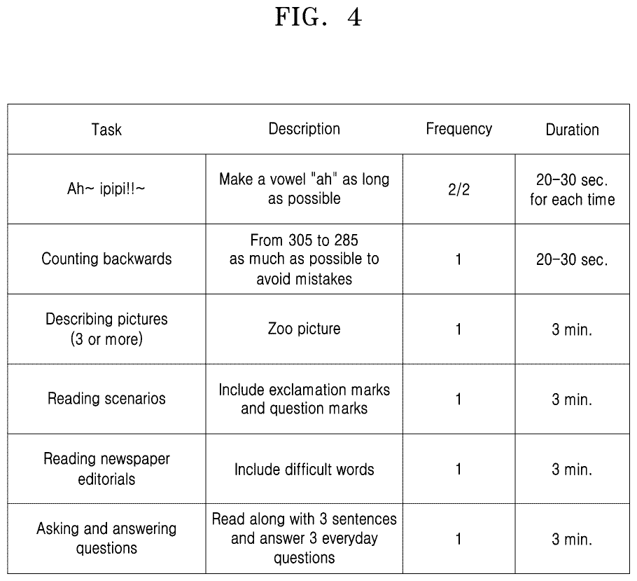

For example, the voice data collection unit 110 may collect voice data generated in a process in which a user (or a patient) performs a list of preset tasks to perform. A user may perform a list of tasks to perform in soundproof facilities or in a noisy environment. The list of preset tasks to perform includes preset tasks to determine voice characteristics, voice diseases, dementia, or depression, by using user's voice data. The list of preset tasks to perform is described in detail in FIG. 4, and is not limited to the disclosure and may be implemented as various embodiments.

The vision data collection unit 120 may collect vision data including a face image of a user who performs a list of preset tasks to perform. The vision data may be used to determine the reliability of voice data. For example, as a result of vision data analysis, a section in which user's face shakes much is determined to have low reliability of voice data and may be excluded from the data to diagnose diseases.

The display unit 130 may be implemented by a monitor that displays a disease diagnosis result according to the present disclosure, and the like, and the storage unit 150 may store the voice data and the vision data. Furthermore, first voice data may be stored in conjunction with patient's mobile phone, and second voice data may be stored, as patient data, in conjunction with electronic medical records (EMR).

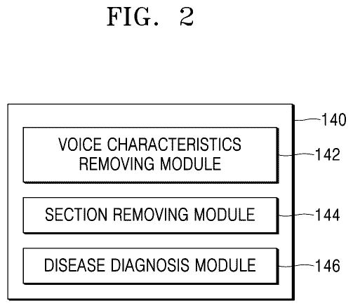

The control unit 140 may analyze user's voice characteristics using the collected voice data, and perform a function to determine diseases, such as dementia, depression, hearing loss, or the like. The control unit 140 is described in detail in FIG. 2.

FIG. 2 is a block diagram for describing a configuration of the control unit 140 according to an embodiment of the present disclosure.

Referring to FIG. 2, the control unit 140 may include a voice characteristic removing module 142, a section removing module 144, and a disease diagnosis module 146.

The voice characteristics removing module 142 may generate second voice data by removing at least one of noise, unique characteristics of voice, or disease characteristics of voice from the first voice data received from the voice data collection unit 110. The voice characteristics removing module 142 may perform a function to generate post-processing data to increase accuracy in disease diagnosis, such as dementia, depression, or the like, by removing noise unrelated to disease diagnosis from the first voice data.

For example, the voice characteristics removing module 142 may remove noise from the first voice data by first measuring noise when the voice data is collected in a noisy environment. Furthermore, the voice characteristics removing module 142 may remove unique characteristics of voice or disease characteristics of voice. The unique characteristics of voice may refer to the characteristics of the tone that a patient possesses. For example, the voice characteristics removing module 142 may remove variables related to age-related changes in tone or voice from the voice data.

The section removing module 144 may determine the reliability of the first voice data or the second voice data by analyzing the vision data transmitted by the vision data collection unit 120. In detail, the section removing module 144 may remove a section in which the reliability of first voice data or second voice data is lower than a preset value, to enable diagnosis of diseases using only voice data with reliability of a certain level, thereby increasing the accuracy in disease diagnosis.

The first voice data or second voice data has analyzable various factors. For example, voice data may have at least one piece of information of a type of voice, glottal attack, resonance, pitch, loudness, or voice quality or timbre. The control unit 140 may diagnose diseases based on AL using the analyzable various factors of voice data.

The disease diagnosis module 146 may diagnose, based on AI, at least one disease of dementia, depression, or hearing loss, using the second voice data.

For example, dementia patients have difficulty naming objects or people due to loss of vocabulary and semantic information from the beginning, and as the disease worsens, pauses between utterances increase. Furthermore, dementia patients use a lot of function words like “this” and “that,” and use long-winded explanations of meanings instead of accurate language, and it takes more time to provide the same positive information. It may be possible to diagnose dementia using voice data by utilizing the linguistic characteristics of the disease.

Furthermore, patients with depression may be diagnosed by analyzing the tone, pitch, loudness, rhythm, and like of voice data to detect clues such as sarcasm and anger that can change the meaning of words when the patients say words expressing positive emotions.

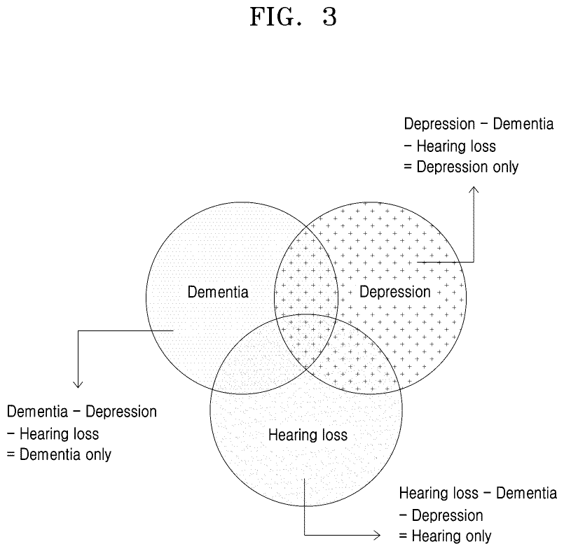

FIG. 3 is a diagram for describing a disease diagnosis method according to an embodiment of the present disclosure.

The disease diagnosis module 146 may diagnose, based on AI, at least one disease of dementia, depression, or hearing loss, using the second voice data.

Dementia, depression, and hearing loss are highly related diseases. The present disclosure may provide an accurate disease diagnosis apparatus suitable for characteristics for each disease by simultaneously performing voice analysis of three diseases with a very high correlation of about 39% to about 50%.

Referring to FIG. 3, the voice data may be classified into data sets for determining respective diseases, and the areas of data sets for determining the respective diseases may overlap each other. For example, a data set for determining dementia may be configured with a data set for dementia only, a data set for dementia and hearing loss, and a data set for dementia, hearing loss, and depression. The present disclosure may increase the accuracy in disease diagnosis using an algorithm for comparing the characteristics of respective groups.

FIG. 4 shows an example of a list of preset tasks to perform according to an embodiment of the present disclosure.

Referring to FIG. 4, the list of preset tasks to perform may include tasks of: first, making a long “ah” sound and making an “ipipi” sound; second, counting backwards; third, describing pictures; fourth, reading scenarios; fifth, reading newspaper editorials; sixth, asking and answering questions; and so forth.

As it is determined based on making a long “ah” sound whether there is any disease or lesion (e.g., false vocal cords or vocal nodules) in the vocal cords, the unique characteristics of user's voice or the disease characteristics of voice may be set. A degree of disease may be determined by making vowels as long as possible, and the unique characteristics of the user's voice or the disease characteristics of the voice may be established and eliminated.

Furthermore, the unique characteristics of voice or the disease characteristics of voice may be set based on making an “ipipi” sound. Voice is the sound produced when air passes through the vocal cords. As the sound may be changed by the pressure created by the air gathering in the subglottic area, i.e., the airway just below the vocal cords, the sound is a task to determine whether the subglottic pressure is normal or not. By performing the task of making an “ah˜ipipi” sound, user' voice characteristics, pitch, and voice quality or timbre may be measured.

Furthermore, cognitive impairment may be determined through a task of counting backwards from 305 to 285. In detail, cognitive impairment may be assessed using accuracy and speed when changing from the 300 s to the 200 s. Furthermore, through the task of counting backwards, glottal attack, pitch, loudness, voice quality or timbre, or resonance information of voice data may be measured.

The list of preset tasks to perform may include a task of seeing and describing a picture. The picture may include a plurality of pictures, and may include pictures that explain operations and pictures that explain nouns. The glottal attack, voice quality or timbre, and the like of voice data may be measured through this test, and used to determine patient' cognitive abilities.

The task of reading scenarios is to identify patients with depression, and may be guided to read scenarios with emotions.

The task of reading newspaper editorials is to identify patients with depression and patients with cognitive impairment, and may be used to determine the speed and pitch at which emotional text and unemotional text are read, how unfamiliar and difficult words are pronounced, and the like.

The task of asking and answering questions is to determine whether a patient has faithfully performed the overall voice data collection task or the level of tension by comparing the level of tension felt in the voice with the patient's usual voice data when a task is given to the patient.

Furthermore, hearing loss may be determined through a list of all tasks to perform.

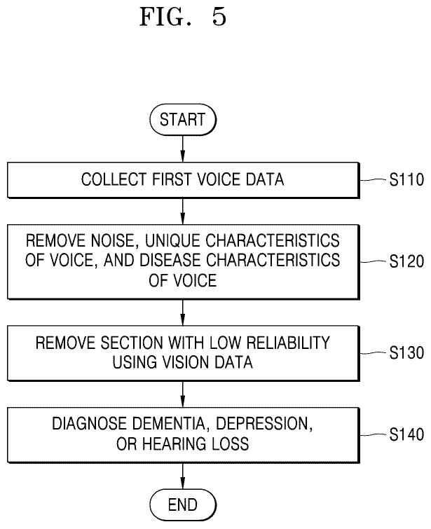

FIG. 5 is a flowchart of an AI-based disease diagnosis method using voice data according to an embodiment of the present disclosure. The disease diagnosis method of FIG. 5 may be performed by the disease diagnosis apparatus described in FIGS. 1 to 4.

In operation S110, the present disclosure may collect first voice data from a user or a patient. Here, the first voice data may be collected based on a list of set tasks to perform. The list of set tasks to perform may include making a long “ah” sound and making an “ipipi” sound, and based on the making a long “ah” sound and the making an “ipipi” sound, the unique characteristics of voice or the disease characteristics of voice may be set. Furthermore, the list of set tasks to perform may include at least one of counting backwards, describing pictures, reading scenarios, or reading newspaper editorials, and based on the list of set tasks to perform, at least one disease of dementia, depression, or hearing loss may be diagnosed.

In operation S120, the present disclosure may generate second voice data by removing, from the first voice data, at least one of noise, the unique characteristics of voice, and the disease characteristics of voice.

The first voice data or the second voice data may include at least one piece of information of a type of voice, glottal attack, resonance, pitch, loudness, or voice quality or timbre.

In operation S130, the present disclosure may include collecting vision data including a face image of a user, determining reliability of the second voice data using the vision data, and removing a section in which the reliability of the second voice data is lower than a preset value.

In operation S140, at least one disease of dementia, depression, or hearing loss may be diagnosed based on AI using the second voice data.

Although exemplary embodiments of the disclosure have been described for illustrative purposes, those having ordinary knowledge in the technical field of the disclosure will appreciate that various modifications, additions and substitutions are possible, without departing from the scope and spirit of the disclosure as disclosed in the accompanying claims. Therefore, the embodiments disclosed in the disclosure are intended to illustrate the scope of the technical idea of the disclosure, and the scope of the technical idea of the disclosure is not limited by the embodiments. The protection scope of the disclosure should be construed based on the accompanying claims, and it should be construed that all of the technical ideas included within the scope equivalent to the claims are included within the right scope of the disclosure.

### INDUSTRIAL APPLICABILITY

The present disclosure relates to an AI-based disease diagnosis method and apparatus using voice data.

1. An AI-based disease diagnosis method using voice data, the method comprising:
collecting first voice data;
generating second voice data by removing at least one of noise, unique characteristics of voice, or disease characteristics of voice from the first voice data; and
diagnosing, based on AI, at least one disease of dementia, depression, or hearing loss, using the second voice data.

2. The disease diagnosis method of claim 1, further comprising:
collecting vision data including a face image of a user;
determining reliability of the second voice data using the vision data; and
removing a section in which the reliability of the second voice data is less than a preset value.

3. The disease diagnosis method of claim 1, wherein
the first voice data or the second voice data comprises at least one piece of information of a type of voice, glottal attack, resonance, pitch, loudness, or voice quality or timbre.

4. The disease diagnosis method of claim 1, wherein
the first data is collected based on a list of set tasks to perform.

5. The disease diagnosis method of claim 4, wherein
the list of set tasks to perform comprises making a long “ah” sound, and
the unique characteristics of voice or the disease characteristics of voice is set based on the making of a long “ah” sound.

6. The disease diagnosis method of claim 4, wherein
the list of set tasks to perform comprises making an “ipipi” sound, and the unique characteristics of voice or the disease characteristics of voice is set based on the making of an “ipipi” sound.

7. The disease diagnosis method of claim 4, wherein
the list of set tasks to perform comprises at least one of counting backwards, describing pictures, reading scenarios, or reading newspaper editorials, and
the at least one disease of dementia, depression, or hearing loss is diagnosed based on the list of set tasks to perform.

8. An AI-based disease diagnosis apparatus using voice data, the apparatus comprising:
a voice data collection unit that collects first voice data; and
a control unit that generates second voice data by removing at least one of noise, unique characteristics of voice, or disease characteristics of voice from the first voice data, and diagnoses, based on AI, at least one disease of dementia, depression, or hearing loss, using the second voice data.

9. The disease diagnosis apparatus of claim 8, further comprising a vision data collection unit that collects vision data including a face image of a user,
wherein the control unit comprises a section removing module that determines reliability of the second voice data using the vision data, and
removes a section in which the reliability of the second voice data is lower than a preset value.

10. The disease diagnosis apparatus of claim 8, wherein
the first voice data or the second voice data comprises at least one piece of information of a type of voice, glottal attack, resonance, pitch, loudness, or voice quality or timbre.

11. The disease diagnosis apparatus of claim 8, wherein
the first data is collected based on a list of set tasks to perform.

12. The disease diagnosis apparatus of claim 11, wherein
the list of set tasks to perform comprises making a long “ah” sound, and
the unique characteristics of voice or the disease characteristics of voice is set based on the making of a long “ah” sound.

13. The disease diagnosis apparatus of claim 11, wherein
the list of set tasks to perform comprises making an “ipipi” sound, and
the unique characteristics of voice or the disease characteristics of voice is set based on the making of an “ipipi” sound.

14. The disease diagnosis apparatus of claim 11, wherein
the list of set tasks to perform comprises at least one of counting backwards, describing pictures, reading scenarios, or reading newspaper editorials, and
the at least one disease of dementia, depression, or hearing loss is diagnosed based on the list of set tasks to perform.
